(19) United States
(12) Patent Application Publication
(10) Pub. No.: US 2026/0283765 A1
(43) Pub. Date: Sep. 24, 2026

(54) FLUID STERILIZATION MODULE AND DENTAL CLEANING DEVICE

(71) Applicant: Xiamen Aihu Dental Technology Co., Ltd., Xiamen (CN)

(72) Inventor: Zhangsen YOU, Xiamen (CN)

(21) Appl. No.: 19/454,282

(22) Filed: Jan. 20, 2026

(30) Foreign Application Priority Data
Mar. 19, 2025 (CN) 202520481265.1

(51) Int. Cl. A61C 17/024; A61L 2/10; C02F 1/32

(52) U.S. Cl. CPC A61C 17/024; A61L 2/10; C02F 1/325; A61C 2204/002; A61L 2202/11; A61L 2202/121; A61L 2202/122; A61L 2202/123; A61L 2202/14; A61L 2202/16; A61L 2202/17; C02F 2201/004; C02F 2201/005; C02F 2201/007; C02F 2201/3227; C02F 2201/326; C02F 2303/04

(57) ABSTRACT

A fluid sterilization module and a dental cleaning device that contains the same. The fluid sterilization module has an outer shell, a control mainboard, a light-blocking sleeve, and a transparent flow tube having one end as a water inlet and another end as a water outlet. The light-blocking sleeve sleeves the transparent flow tube, and the outer shell sleeves the light-blocking sleeve. The control mainboard is mounted on the outer shell and contains sterilization lamps. Light-transmitting zones corresponding to the sterilization lamps are provided on the light-blocking sleeve.

### TECHNICAL FIELD

The present invention relates to the technical field of dental cleaning devices, particularly to a fluid sterilization module and a dental cleaning device.

### BACKGROUND OF THE INVENTION

With the improvement in living standards, the emphasis on oral health has also increased. Currently, brushing teeth is the most common method for oral cleaning, but toothbrushes cannot clean the tiny gaps between teeth, leading to the accumulation of plaque and causing dental and gum diseases. As a result, dental cleaning devices, more commonly known as oral irrigators, have emerged, which use pulsed water jets to clean teeth and interdental spaces, thereby ensuring oral health.

However, residual liquid in dental cleaning devices after use can breed bacteria over time during storage and operation. These bacteria may enter the oral cavity during cleaning, thus posing a threat to oral health. Some dental cleaning devices incorporate ultraviolet (UV) sterilization lamps in their water tanks for disinfection, but the water tanks, due to their great sizes, usually cannot be effectively disinfected by using only one or two UV lamps. For example, CN115211988A discloses an oral cleaning device with a sterilizer that performs instant sterilization of the fluid flowing in the flow path. However, according to the structural design of this prior art patent application, the circuit board is separated from the sterilizer and only connected to the sterilizer through wires to prevent water leakage damage at a position of the flow path where the sterilizer is mounted, however, such structural design leads to complex installation, fragile wiring, and poor reliability.

### BRIEF SUMMARY OF THE INVENTION

In view of the aforesaid disadvantages now present in the prior art, the present invention provides a fluid sterilization module and a dental cleaning device. The present invention is reliable and enables quick and effective sterilization and disinfection. Use of the dental cleaning device is ensured to be safe, and the user's experience is therefore enhanced.

To attain the above object of the present invention, the present invention provides the following technical solutions:

A fluid sterilization module, comprising an outer shell, a control mainboard, a light-blocking sleeve, and a transparent flow tube;
one end of the transparent flow tube is a water inlet of the fluid sterilization module; another end of the transparent flow tube is a water outlet of the fluid sterilization module;
the light-blocking sleeve sleeves around a peripheral side of the transparent flow tube, and the outer shell sleeves around a peripheral side of the light-blocking sleeve; the control mainboard is installed on the outer shell and comprises a plurality of sterilization lamps; the light-blocking sleeve is provided with a plurality of light-transmitting zones corresponding to the plurality of sterilization lamps to allow the sterilization lamps to irradiate fluid inside the transparent flow tube for sterilization.

Further, the transparent flow tube is an elongated tube extending vertically; the plurality of sterilization lamps are positioned along an axial direction of the transparent flow tube and being spaced apart from one another.

Further, the transparent flow tube is made of high-temperature-resistant glass;
the plurality of light-transmitting zones of the light-blocking sleeve are through holes penetrating through the light-blocking sleeve.

Further, one side of the outer shell comprises an installation slot in which the control mainboard is mounted; the installation slot comprises a plurality of lamp holes in communication with an inner cavity of the outer shell; the plurality of sterilization lamps are inserted through the plurality of lamp holes; the light-blocking sleeve is positioned in the inner cavity.

Further, the outer shell comprises an upper opening in communication with an inner cavity of the outer shell, and a bottom of the inner cavity forms an installation stepped portion; the transparent flow tube and the light-blocking sleeve are inserted into the inner cavity from the upper opening; lower ends of the transparent flow tube and the light-blocking sleeve abut against the installation stepped portion; the installation stepped portion comprises a lower hole connected to an external environment.

Further, the outer shell comprises an upper opening in communication with an inner cavity of the outer shell and in communication with the water outlet of the fluid sterilization module at said another end of the transparent flow tube; the upper opening is connecting with an adapter; the adapter has a check valve that allows one-way water flow from the fluid sterilization module to an external environment.

A dental cleaning device, comprising a dental cleaning device body, a pump spray module, said fluid sterilization module, and a nozzle;
the dental cleaning device body comprises an equipment chamber and a water storage chamber independent from each other; the pump spray module and the fluid sterilization module are installed in the equipment chamber; the water storage chamber is configured to store fluid for dental cleaning;
the nozzle is detachably mounted at one end of the dental cleaning device body and connected to a water outlet of the pump spray module; a water inlet of the pump spray module is connected to the water outlet of the fluid sterilization module, and the water inlet of the fluid sterilization module is connected to the water storage chamber; the pump spray module draws the fluid from the water storage chamber, through the fluid sterilization module in which the fluid is sterilized, and then ejects the fluid through the nozzle.

Further, the water inlet of the fluid sterilization module is connected to one end of a water inlet tube; another end of the water inlet tube is inserted into the water storage chamber; said another end of the water inlet tube has an inclined section that extends to an inner bottom surface of the water storage chamber; an edge of a free end of the inclined section touches the inner bottom surface of the water storage chamber; the free end of the inclined section is positioned right below the nozzle; the water outlet of the fluid sterilization module is connected to one end of an adapter; another end of the adapter is connected to the water inlet of the pump spray module.

Further, the pump spray module comprises a housing, a spray pump, a motor, a dental cleaning control board, and a battery; the spray pump and the motor are installed inside the housing; the motor drives the spray pump to operate; a water inlet of the spray pump is connected to the water outlet of the fluid sterilization module, and a water outlet of the spray pump is connected to the nozzle; the motor and the battery are electrically connected to the dental cleaning control board; the battery is configured to power the motor; the dental cleaning control board controls the motor to operate; the dental cleaning control board also comprises a charging port for charging the battery;
the dental cleaning device body comprises an upper cover; the equipment chamber and water storage chamber are arranged side by side inside the dental cleaning device body; the upper cover seals top sides of both the equipment chamber and the water storage chamber; one side of the upper cover comprises a water inlet hole in communication with the water storage chamber; the water inlet hole is removably covered by a sealing cap; a retaining ring is provided inside the water storage chamber below the water inlet hole to hold and store the nozzle inside the water storage chamber when the nozzle is not in use.

According to the technical solutions given above, when fluid flows from the water inlet of the fluid sterilization module into the transparent flow tube, the sterilization lamps can instantly sterilize the fluid passing through the transparent flow tube. Further, as the light-blocking sleeve sleeves around the peripheral side of the transparent flow tube, the light-blocking sleeve prevents scattering of sterilization light from the sterilization lamps, thereby enhancing the sterilization effect. By sleeving the transparent flow tube and the light-blocking sleeve within the outer shell, the control mainboard is separated from the transparent flow tube in which water flows to improve safety and preventing damage to the control mainboard in case of water leakage from the transparent flow tube, thereby enhancing product reliability.

According to the dental cleaning device of the present invention, the pump spray module draws water from the water storage chamber, through the fluid sterilization module in which the water is sterilized, and then ejects the water through the nozzle. This ensures that clean water being sterilized and disinfected is used for cleaning the teeth and the gaps therebetween, reducing the risk of infection during use of the dental cleaning device, and hence improving safety.

### BRIEF DESCRIPTION OF THE DRAWINGS

FIG. 1 is a perspective view of an embodiment of the dental cleaning device;

FIG. 2 is an exploded view of an embodiment of the dental cleaning device;

FIG. 3 is a top view of an embodiment of the dental cleaning device;

FIG. 4 is a cross-sectional view taken along line A-A in FIG. 3;

FIG. 5 is a cross-sectional view taken along line B-B in FIG. 3;

FIG. 6 is an exploded view of the fluid sterilization module;

FIG. 7 is an exploded view of the pump spray module;

FIG. 8 is a perspective view of the dental cleaning device with an alternative outer shape;

FIG. 9 is a perspective view of the dental cleaning device with yet another outer shape.

### REFERENCE NUMERALS

Dental cleaning device body 1, equipment chamber 11, water storage chamber 12, upper cover 13, water inlet hole 131, nozzle mounting hole 132, lower cover 14, sealing cap 15, retaining ring 16;
Pump spray module 2, housing 21, spray pump 22, motor 23, dental cleaning control board 24, charging port 241, control panel 242, battery 25, water outlet 201 of the pump spray module, water inlet 202 of the pump spray module;
Fluid sterilization module 3, outer shell 31, installation slot 311, lamp hole 312, inner cavity 313 of the outer shell, upper opening 314, installation stepped portion 315, lower hole 316 of the outer shell, clamping groove 317, clamping seat 318, control mainboard 32, sterilization lamp 321, light-blocking sleeve 33, light-transmitting zone 331, transparent flow tube 34, lower sealing connector 35, upper sealing connector 36, water outlet 301 of the fluid sterilization module, water inlet 302 of the fluid sterilization module;
Nozzle 4, water inlet tube 5, inclined section 51, adapter 6, clamping block 61, check valve 7, check piston 71, piston spring 72.

### DETAILED DESCRIPTION OF THE INVENTION

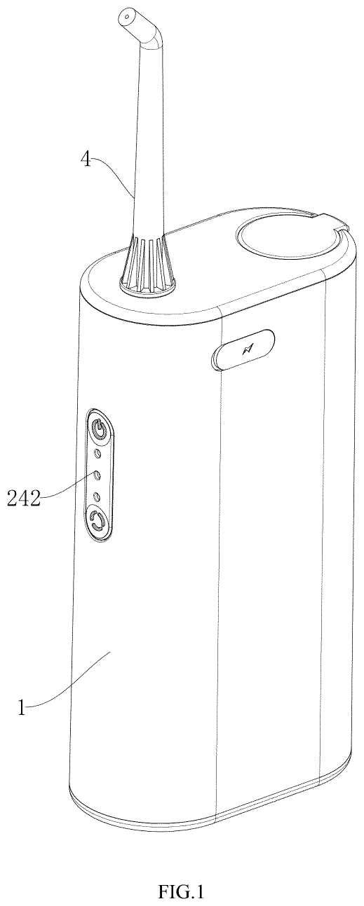

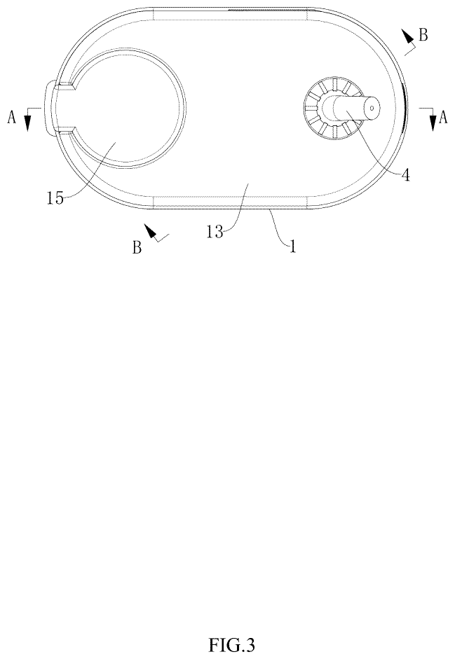

As shown in FIGS. 1 to 5, an embodiment of the dental cleaning device comprises a dental cleaning device body 1, a pump spray module 2, a fluid sterilization module 3, and a nozzle 4.

The dental cleaning device body 1 comprises an equipment chamber 11 and a water storage chamber 12 independent from each other; the pump spray module 2 and the fluid sterilization module 3 are installed in the equipment chamber 11; the water storage chamber 12 is configured to store clean water or other liquids for dental cleaning.

The nozzle 4 is detachably mounted at one end of the dental cleaning device body 1 and connected to a water outlet 201 of the pump spray module 2; a water inlet 202 of the pump spray module 2 is connected to a water outlet 301 of the fluid sterilization module 3, and a water inlet 302 of the fluid sterilization module 3 is connected to the water storage chamber 12. The pump spray module 2 draws water from the water storage chamber 12, through the fluid sterilization module 3 for sterilization, and then ejects the water through the nozzle 4. This ensures that clean water being sterilized and disinfected is used for cleaning the teeth and the gaps therebetween, reducing the risk of infection during use of the dental cleaning device, and hence improving safety.

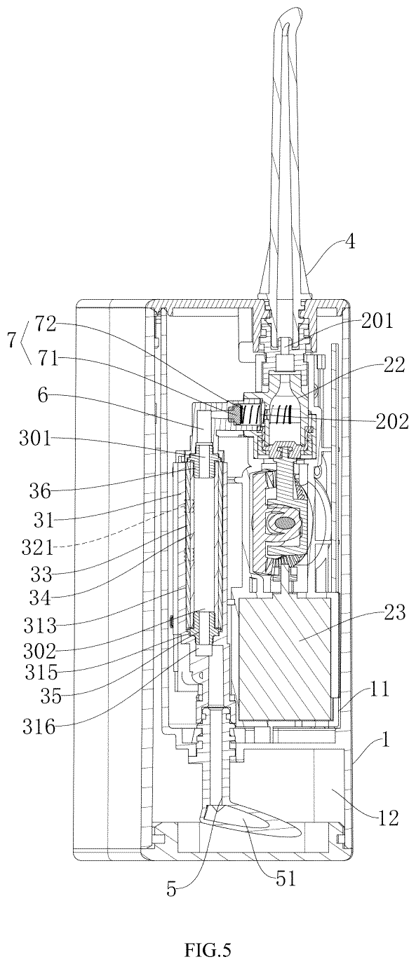

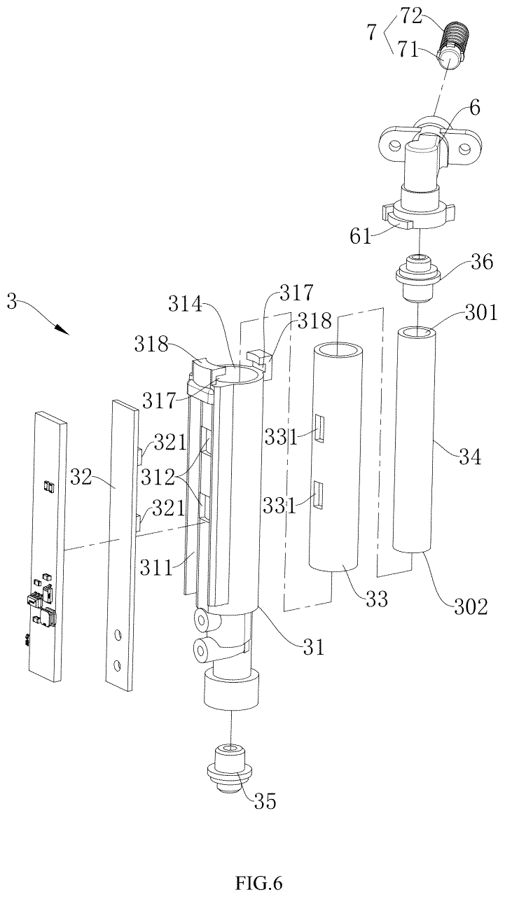

As shown in FIGS. 5 and 6, the fluid sterilization module 3 comprises an outer shell 31, a control mainboard 32, a light-blocking sleeve 33, and a transparent flow tube 34.

The transparent flow tube 34 may be an elongated tube extending vertically. One end of the transparent flow tube 34 serves as the water inlet 302 of the fluid sterilization module 3 and is connected to one end of a water inlet tube 5 via a lower sealing connector 35. Another end of the water inlet tube 5 is inserted into the water storage chamber 12. Another end of the transparent flow tube 34 serves as the water outlet 301 of the fluid sterilization module 3 and is connected to one end of an adapter 6 via an upper sealing connector 36. Another end of the adapter 6 is connected to the water inlet 202 of the pump spray module 2.

The light-blocking sleeve 33 sleeves around a peripheral side of the transparent flow tube 34, and the outer shell 31 sleeves around a peripheral side of the light-blocking sleeve 33. The control mainboard 32 is installed on the outer shell 31 and comprises a plurality of sterilization lamps 321. The light-blocking sleeve 33 is provided with a plurality of light-transmitting zones 331 corresponding to the plurality of sterilization lamps 321 to allow the sterilization lamps 321 to irradiate the water inside the transparent flow tube 34 for sterilization.

Since the transparent flow tube 34 is located between the water storage chamber 12 and the pump spray module 2, when water is drawn from the water storage chamber 12 and flows into the transparent flow tube 34, the sterilization lamps 321 can instantly sterilize the water passing through the transparent flow tube 34. Further, as the light-blocking sleeve 33 sleeves around the peripheral side of the transparent flow tube 34, the light-blocking sleeve 33 prevents scattering of sterilization light from the sterilization lamps 321, and thereby enhancing the sterilization effect.

The transparent flow tube 34 may be an elongated tube, and the plurality of sterilization lamps 321 may be positioned along an axial direction of the transparent flow tube 34 and being spaced apart from one another. This ensures thorough sterilization through a longer time even at a high water flow rate so as to enhance the sterilization effect. In this embodiment, two dual-wavelength UV sterilization lamps 321 are used as examples for enhanced sterilization, capable of killing specific bacteria. Alternatively, single-wavelength sterilization lamps may also be used.

Specifically, one side of the outer shell 31 may comprise an installation slot 311 for mounting the control mainboard 32. The installation slot 311 comprises a plurality of lamp holes 312 in communication with an inner cavity 313 of the outer shell 31; the plurality of sterilization lamps 321 are inserted through the plurality of lamp holes 312. The light-blocking sleeve 33 is positioned in the inner cavity 313. The plurality of light-transmitting zones 331 of the light-blocking sleeve 33 may be through holes penetrating through the light-blocking sleeve 33 to allow the plurality of sterilization lamps 321 to pass through and irradiate the transparent flow tube 34. The transparent flow tube 34 made of high-temperature-resistant glass is installed inside the light-blocking sleeve 33.

By sleeving the transparent flow tube 34 and the light-blocking sleeve 33 within the outer shell 31, the control mainboard 32 is separated from the transparent flow tube 34 in which water flows to improve safety and preventing damage to the control mainboard 32 in case of water leakage from the transparent flow tube 34, thereby enhancing product reliability.

The outer shell 31 comprises an upper opening 314 in communication with the inner cavity 313, and a bottom of the inner cavity 313 forms an installation stepped portion 315. The transparent flow tube 34 and the light-blocking sleeve 33 can be inserted into the inner cavity 313 from the upper opening 314; lower ends of the transparent flow tube 34 and the light-blocking sleeve 33 abut against the lower sealing connector 35 and rest on the installation stepped portion 315. The installation stepped portion 315 comprises a lower hole 316 connected to an external environment to facilitate assembly. The transparent flow tube 34 can be connected to the water inlet tube 5 via the lower sealing connector 35 at the lower hole 316.

The upper opening 314 of the outer shell 31 may comprise two clamping seats 318 with two side clamping grooves 317 respectively. The adapter 6 may have an inverted L shape, with two clamping blocks 61 at around said one end of the adapter 6. The adapter 6 can be quickly clamped to the upper opening 314 by rotating the adapter 6 during installation to the upper opening 314 such that the clamping blocks 61 slide into the clamping grooves 317. The upper sealing connector 36 is placed between the upper opening 314 and said one end of the adapter 6 to achieve a sealing effect between the upper opening 314 and said one end of the adapter 6.

In this embodiment, the fluid sterilization module 3 and the pump spray module 2 are arranged side by side. Said another end of the adapter 6 defined as a right opening of the adapter 6 is connected to the water inlet 202 of the pump spray module 2.

The right opening of the adapter 6 may also be connected to a check valve 7 to ensure one-way water flow from the sterilization module 3 to the pump spray module 2, preventing backflow of water from the nozzle 4. The check valve 7 comprises a check piston 71 and a piston spring 72.

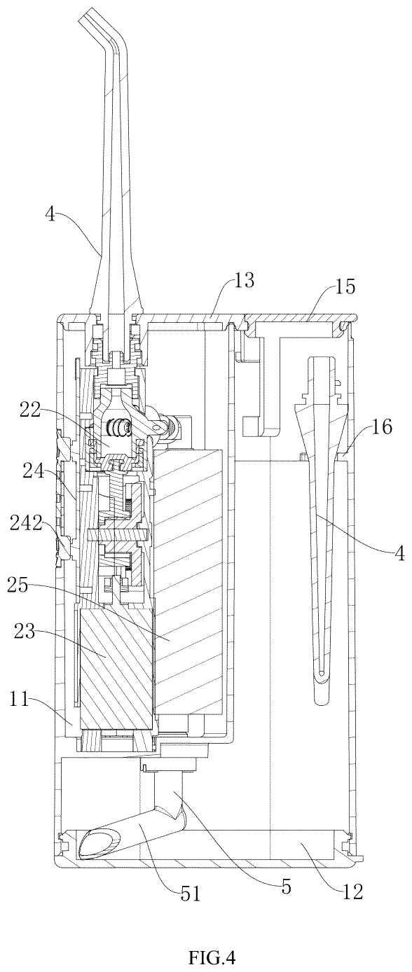

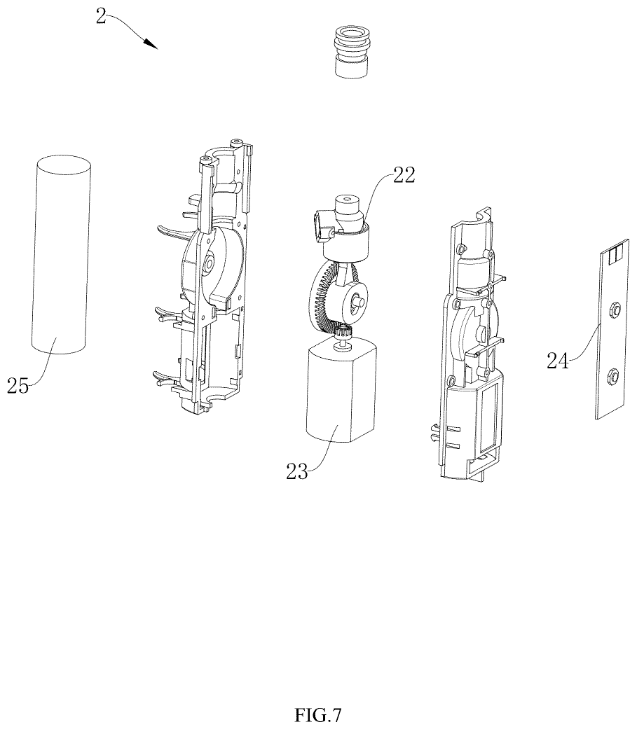

As shown in FIGS. 4, 5, and 7, the pump spray module 2 comprises a housing 21, a spray pump 22, a motor 23, a dental cleaning control board 24, and a battery 25. The spray pump 22 and the motor 23 are installed inside the housing 21; the motor 23 drives the spray pump 22 to operate. A water inlet of the spray pump 22 is connected to the right opening of the adapter 6, and a water outlet of the spray pump 22 is connected to the nozzle 4. The battery 25 and the dental cleaning control board 24 are installed on two opposite sides of the housing 21 respectively. The motor 23 and the battery 25 are electrically connected to the dental cleaning control board 24. The battery 25 is configured to power the motor 23; the dental cleaning control board 24 controls the motor 23 to operate; the dental cleaning control board 24 also comprises a charging port 241 for charging the battery 25.

The control mainboard 32 is also electrically connected to the dental cleaning control board 24, allowing the dental cleaning control board 24 to control the plurality of sterilization lamps 321. The dental cleaning control board 24 has a control panel 242 exposed on an outer surface of the dental cleaning device body 1.

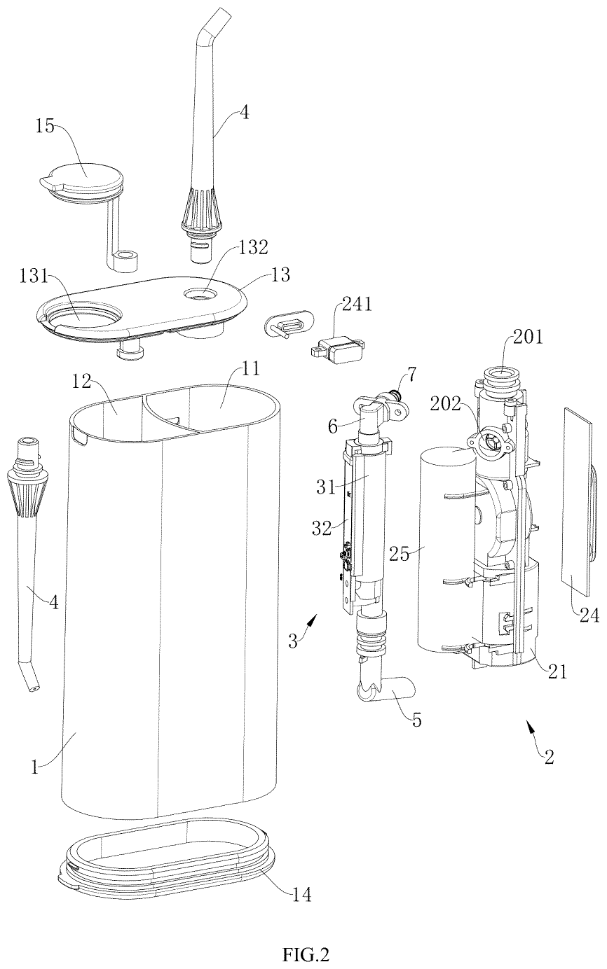

As shown in FIG. 2, according to an embodiment of the present invention, the dental cleaning device body 1 comprises an upper cover 13 and a lower cover 14 to cover an upper side and a lower side of the dental cleaning device body 1 respectively. The equipment chamber 11 and water storage chamber 12 are arranged side by side inside the dental cleaning device body 1. The upper cover 13 seals top sides of both the equipment chamber 11 and the water storage chamber 12; one side of the upper cover 13 comprises a water inlet hole 131 in communication with the water storage chamber 12; the water inlet hole 131 is removably covered by a sealing cap 15 to facilitate water replenishment into the water storage chamber 12. A retaining ring 16 is provided inside the water storage chamber 12 below the water inlet hole 131 to hold and store the nozzle 4 inside the water storage chamber 12 when the nozzle 4 is not in use, thereby reducing product size for easy storage and portability.

Another side of the upper cover 13 also comprises a nozzle mounting hole 132; the nozzle 4 is detachably installed in the nozzle mounting hole 132.

The lower cover 14 seals the lower side of the dental cleaning device body 1, in particularly a bottom of the water storage chamber 12.

As shown in FIGS. 4 and 5, said one end of the water inlet tube 5 extends into the equipment chamber 11 to connect with the fluid sterilization module 3. Said another end of the water inlet tube 5 has an inclined section 51 that extends to an inner bottom surface of the water storage chamber 12; an edge of a free end of the inclined section 51 touches the inner bottom surface of the water storage chamber 12; the free end of the inclined section 51 is positioned right below the nozzle 4.

When the user holds the dental cleaning device during use, the nozzle 4 typically extends into the oral cavity at a slight tilt. As the water in the water storage chamber 12 is nearly depleted, residual water accumulates in a corner of the water storage chamber 12 directly beneath the nozzle 4. Many existing dental cleaning devices in the market fail to fully draw out the water from this corner, leaving approximately one-third of the water unused. In this embodiment, the water inlet tube 5 is equipped with an inclined section 51 that touches the inner bottom surface of the water storage chamber 12, with its free end positioned directly below the nozzle 4 where the residual water precisely accumulates. As a result, the inclined section 51 ensures complete drainage of water in the water storage chamber 12, eliminating stagnant water and preventing bacterial growth.

In summary, this embodiment of the present invention provides a dental cleaning device featuring a fluid sterilization module 3 installed between the water storage chamber 12 and the pump spray module 2. The spray pump 22 of the pump spray module 2 draws water from the water storage chamber 12 into the transparent flow tube 34 of the sterilization module 3, where the sterilization lamps 321 instantly sterilize and disinfect the water. This guarantees the cleanliness of the water ejected from the nozzle 4, delivering highly efficient and reliable sterilization. Additionally, the water inlet tube 5, with its inclined section 51, ensures complete drainage of water in the water storage chamber 12.

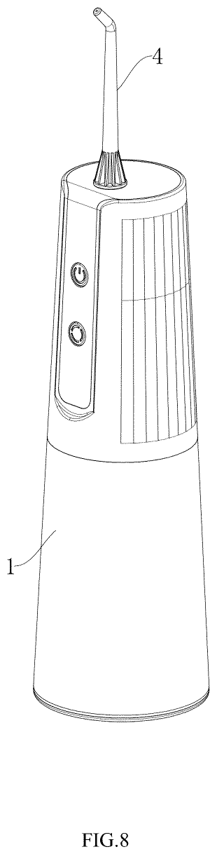

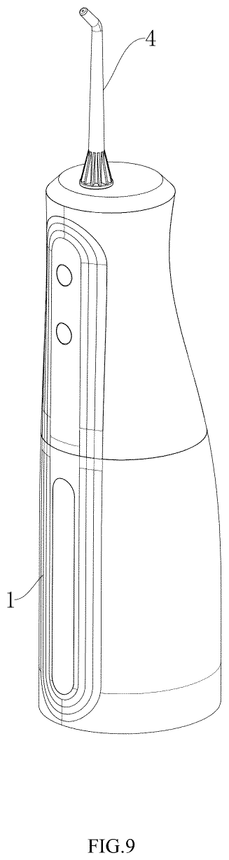

As further illustrated in FIGS. 8 and 9, the dental cleaning device body 1 may have different outer shapes.

In this embodiment, the dental cleaning device body 1 may be made of PP, ABS, PET, or similar materials with lubricating additives to achieve a lotus effect, preventing water droplets from adhering to the inner walls of the water storage tank 12 and inhibiting bacterial growth, thereby enhancing hygiene and safety.

What is claimed is:

1. A fluid sterilization module, comprising:
an outer shell, a control mainboard, a light-blocking sleeve, and a transparent flow tube;
one end of the transparent flow tube is a water inlet of the fluid sterilization module; another end of the transparent flow tube is a water outlet of the fluid sterilization module;
the light-blocking sleeve sleeves around a peripheral side of the transparent flow tube, and the outer shell sleeves around a peripheral side of the light-blocking sleeve; the control mainboard is installed on the outer shell and comprises a plurality of sterilization lamps; the light-blocking sleeve is provided with a plurality of light-transmitting zones corresponding to the plurality of sterilization lamps to allow the sterilization lamps to irradiate fluid inside the transparent flow tube for sterilization.

2. The fluid sterilization module of claim 1, wherein the transparent flow tube is an elongated tube extending vertically; the plurality of sterilization lamps are positioned along an axial direction of the transparent flow tube and being spaced apart from one another.

3. The fluid sterilization module of claim 1, wherein the transparent flow tube is made of high-temperature-resistant glass; the plurality of light-transmitting zones of the light-blocking sleeve are through holes penetrating through the light-blocking sleeve.

4. The fluid sterilization module of claim 1, wherein one side of the outer shell comprises an installation slot in which the control mainboard is mounted; the installation slot comprises a plurality of lamp holes in communication with an inner cavity of the outer shell; the plurality of sterilization lamps are inserted through the plurality of lamp holes; the light-blocking sleeve is positioned in the inner cavity.

5. The fluid sterilization module of claim 1, wherein the outer shell comprises an upper opening in communication with an inner cavity of the outer shell, and a bottom of the inner cavity forms an installation stepped portion; the transparent flow tube and the light-blocking sleeve are inserted into the inner cavity from the upper opening; lower ends of the transparent flow tube and the light-blocking sleeve abut against the installation stepped portion; the installation stepped portion comprises a lower hole connected to an external environment.

6. The fluid sterilization module of claim 1, wherein the outer shell comprises an upper opening in communication with an inner cavity of the outer shell and in communication with the water outlet of the fluid sterilization module at said another end of the transparent flow tube; the upper opening is connected with an adapter; the adapter has a check valve that allows one-way water flow from the fluid sterilization module to an external environment.

7. A dental cleaning device, comprising said fluid sterilization module of claim 1, and further comprising a dental cleaning device body, a pump spray module, and a nozzle;
the dental cleaning device body comprises an equipment chamber and a water storage chamber independent from each other; the pump spray module and the fluid sterilization module are installed in the equipment chamber; the water storage chamber is configured to store fluid for dental cleaning;
the nozzle is detachably mounted at one end of the dental cleaning device body and connected to a water outlet of the pump spray module; a water inlet of the pump spray module is connected to the water outlet of the fluid sterilization module, and the water inlet of the fluid sterilization module is connected to the water storage chamber; the pump spray module draws the fluid from the water storage chamber, through the fluid sterilization module in which the fluid is sterilized, and then ejects the fluid through the nozzle.

8. The dental cleaning device of claim 7, wherein the water inlet of the fluid sterilization module is connected to one end of a water inlet tube; another end of the water inlet tube is inserted into the water storage chamber; said another end of the water inlet tube has an inclined section that extends to an inner bottom surface of the water storage chamber; an edge of a free end of the inclined section touches the inner bottom surface of the water storage chamber; the free end of the inclined section is positioned right below the nozzle; the water outlet of the fluid sterilization module is connected to one end of an adapter; another end of the adapter is connected to the water inlet of the pump spray module.

9. The dental cleaning device of claim 7, wherein the pump spray module comprises a housing, a spray pump, a motor, a dental cleaning control board, and a battery; the spray pump and the motor are installed inside the housing; the motor drives the spray pump to operate; a water inlet of the spray pump is connected to the water outlet of the fluid sterilization module, and a water outlet of the spray pump is connected to the nozzle; the motor and the battery are electrically connected to the dental cleaning control board; the battery is configured to power the motor; the dental cleaning control board controls the motor to operate; the dental cleaning control board also comprises a charging port for charging the battery;
the dental cleaning device body comprises an upper cover; the equipment chamber and water storage chamber are arranged side by side inside the dental cleaning device body; the upper cover seals top sides of both the equipment chamber and the water storage chamber; one side of the upper cover comprises a water inlet hole in communication with the water storage chamber; the water inlet hole is removably covered by a sealing cap; a retaining ring is provided inside the water storage chamber below the water inlet hole to hold and store the nozzle inside the water storage chamber when the nozzle is not in use.
